(19) United States
(12) Patent Application Publication
(10) Pub. No.: US 2012/0330193 A1
(43) Pub. Date: Dec. 27, 2012

(54) DEVICES AND METHODS FOR MEASURING JOINT ROTATION OF AN OBJECT

(75) Inventor: Tik-Pui Daniel Fong, Hong Kong (CN)

(73) Assignee: THE CHINESE UNIVERSITY OF HONG KONG, Hong Kong (CN)

(21) Appl. No.: 13/529,889

(22) Filed: Jun. 21, 2012

Related U.S. Application Data
(60) Provisional application No. 61/499,690, filed on Jun. 22, 2011.

(51) Int. Cl. A61B 5/11

(52) U.S. Cl. 600/595

(57) ABSTRACT

A device and method for measuring joint rotation of an object, which provides an objective quantification of a laxity of the joint, especially in external and internal rotation. The device for measuring rotation of a first joint of an object comprises an orthosis for immobilizing a second joint of the object, the second joint being connected to the first joint by a bone of the object; a torque sensor fixed to the orthosis for monitoring a value of torque applied to the second joint, the applied torque being transferred to the first joint via the bone; and a motion sensor for tracking a rotational displacement of the bone occurring under the applied torque.

### CROSS REFERENCE TO RELATED APPLICATIONS

This application claims the benefit of priority under 35 U.S.C. §119(e) of U.S. Provisional Patent Application No. 61/499,690 filed on Jun. 22, 2011, which is explicitly incorporated by reference in its entirety.

### TECHNICAL FIELD

The present application relates to devices and methods for measuring joint rotation of an object, in particular, devices and methods for measuring joint rotation of a person who has suffered from a joint injury.

### BACKGROUND

In the past, assessment of joint laxity has replied upon clinical examination, which is subjective.

### SUMMARY

The present application relates to a device and a method for measuring joint rotation of an object, which would provide an objective quantification of a laxity of the joint, especially in external and internal rotation.

According to an aspect of the application, provided is a device for measuring rotation of a first joint of an object, comprising: an orthosis for immobilizing a second joint of the object, the second joint being connected to the first joint by a bone of the object; a torque sensor fixed to the orthosis for monitoring a value of torque applied to the second joint, the applied torque being transferred to the first joint via the bone; and a motion sensor for tracking a rotational displacement of the bone occurring under the applied torque.

According to another aspect of the application, provided is method for measuring rotation of a first joint of an object, comprising: immobilizing a second joint of the object, the second joint being connected to the first joint by a bone of the object; monitoring a value of torque applied to the second joint, the applied torque being transferred to the first joint via the bone; and tracking a rotational displacement of the bone under the applied torque.

### BRIEF DESCRIPTION OF THE DRAWINGS

FIG. 1 is an illustrative block diagram of the device for measuring joint rotation according to an embodiment of the present application;

FIG. 2 is an illustrative flow chart of the method for measuring joint rotation according to an embodiment of the present application;

FIGS. 3(a) and 3(b) illustrate an example of the device of FIG. 1;

FIG. 4 is an example of a real application of the device of FIGS. 3(a); and

FIG. 5 is an example of a cadaver test of the device of FIG. 3(a).

### DETAILED DESCRIPTION OF THE PREFERRED EMBODIMENTS

Hereinafter, embodiments according to the present application will be described with reference to the accompanying drawings.

A device 10 for measuring rotation of a first joint of an object shown is shown in FIG. 1. As shown, the device 10 comprises an orthosis 11, a torque sensor 12 and a motion sensor 13.

The orthosis 11 may be a common orthotic device that is used for immobilizing a second joint of the object, which is connected with the first joint by a bone of the object.

The torque sensor 12 is fixed to the orthosis for monitoring a value of torque applied to the object, in particular, the immobilized second joint. Since the second joint is immobilized, the applied torque is transferred to the first joint via the bone therebetween. The torque may be applied in a direction such that the first joint rotates inwardly or outwardly with the bone. The torque sensor may be fixed to a bottom of the orthosis. The torque sensor may be a load cell.

Motion sensor 13 is arranged for tracking a rotational displacement of the bone occurring under the applied torque. The motion sensor may be fixed to the torque sensor. The motion sensor may also be arranged so that a longitudinal axis of the motion sensor is along the bone.

In an example, the motion sensor is an electromagnetic tracking sensor and may have an acquisition frequency of up to 120 Hz. Thus, tracking data with high accuracy may be obtained in time. The obtained tracking data may represent a laxity (an extent of rotation) of the first joint under a certain torque, and may be output to an external device.

According to an embodiment, a handle may be fixed to the orthosis for applying the torque. For example, a handle bar may be fixed to the torque sensor so that the torque may be easily applied.

In an example, the first joint is a knee joint, the second joint is an ankle joint, and the bone connecting the first and second joints is a tibia.

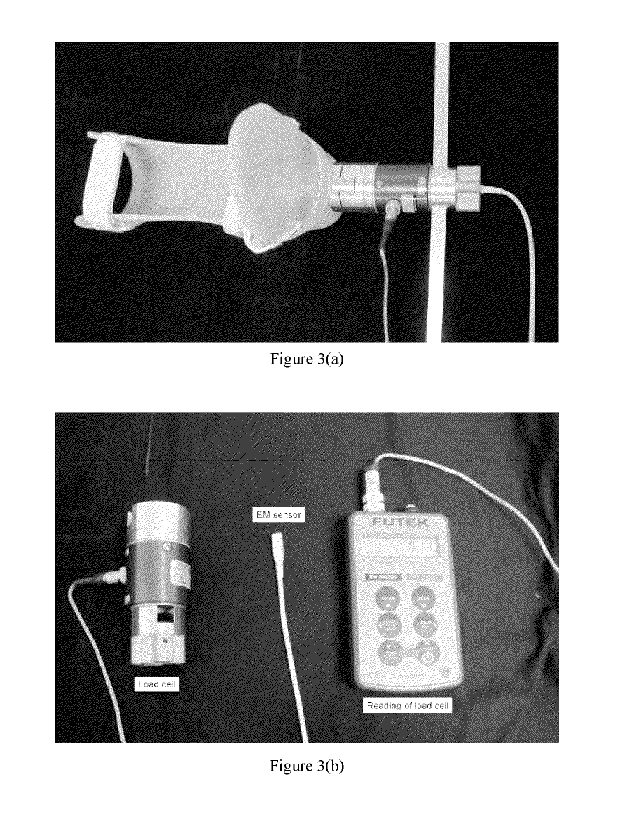

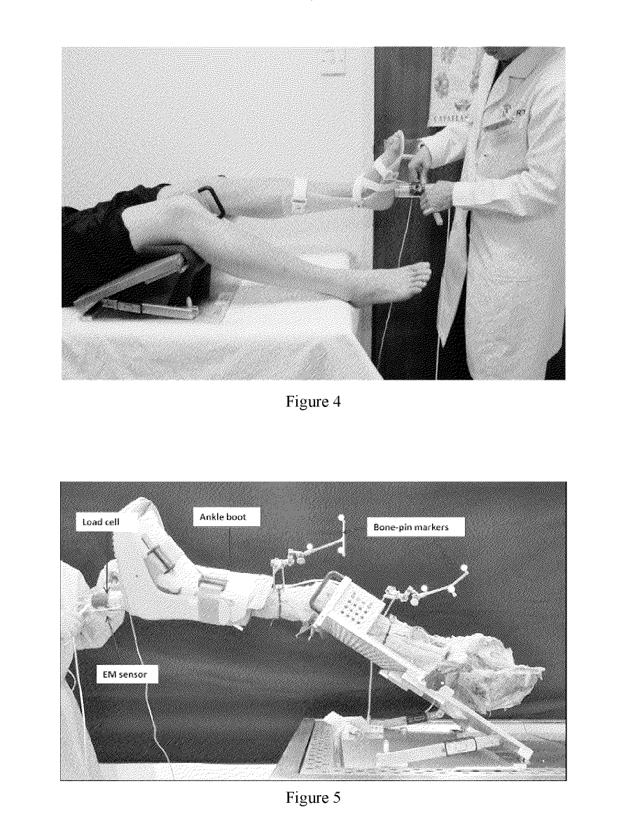

An example of the device is shown in FIGS. 3(a) and 3(b). A real application of the device is shown in FIG. 4. A cadaver test of the device is shown in FIG. 5.

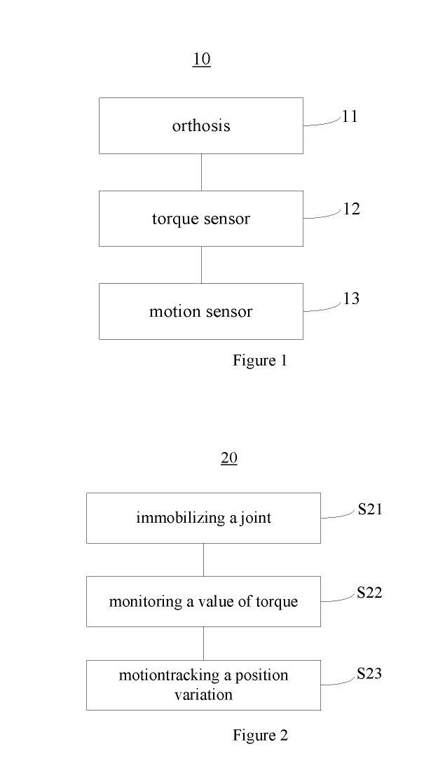

Hereinafter, a method 20 for measuring rotation of a first joint of an object will be described with an assumption that the first joint is a knee joint, the second joint is an ankle joint, and the bone connecting the first and second joints is a tibia with reference to FIG. 2 in combination with FIGS. 3(a), 3(b), 4 and 5.

In FIGS. 4 and 5, the orthosis takes the form of an ankle boot, which immobilizes the ankle joint of the object. The torque sensor takes the form of a load cell, which is mounted to a heel region of the ankle boot. The motion sensor takes the form of an electromagnetic motion sensor, which is attached to the load cell. The longitudinal axis of the motion sensor is along the tibia's axis of rotation. The tracking data obtained by the motion sensor are output to an external device such as a laptop computer. According to the method, the ankle joint is immobilized at step S21. After a torque is applied to the ankle boot, at step S22, a value of the applied torque is monitored. The applied torque transfers to the knee joint via the tibia. Under the torque, the tibia is moved. Upon the movement, a rotational displacement of the tibia is obtained by the motion sensor. Based on the obtained data, the laxity of the knee joint under each torque are determined. Since the torque may be applied in a direction such that the knee joint rotates inwardly or outwardly, the extent of rotation of the knee joint under each torque is obtained.

A cadaveric experiment was performed to evaluate the validity and reliability of the present application based on the configuration shown in FIG. 5. Five cadaver lower limbs were obtained. A bone-pin based motion analysis system was employed as the golden standard. After attaching marker, i.e., motion trackers, to the femur and tibia, two research staffs conducted the test by applying internal and external rotation torque from 0 to 7N to the tibia of the cadaver. The tests were performed with the knee flexed at 30 degree and 90 degree. Data were analyzed using intra-class correlation (ICC) and standard error of measurement (SEM). Results in the following two Tables demonstrated excellent validity and reliability in the device.

TABLE ONE
Intra-class correlation.

Intra ICC

| | Tester 1 | Tester 2 | Inter ICC |
|---|---|---|---|
| 30 deg | 0.994 | 0.996 | 0.996 |
| 90 deg | 0.979 | 0.992 | 0.931 |

TABLE TWO
Standard error of measurement.

SEM

| | External rotation | Internal rotation |
|---|---|---|
| | (deg) | (deg) |
| 30 Deg | | |
| 3 Nm | 0.5074 | 0.3256 |
| 5 Nm | 0.2225 | 0.6079 |
| 7 Nm | 0.2037 | 1.0592 |
| 90 Deg | | |
| 3 Nm | 0.5298 | 0.3273 |
| 5 Nm | 0.7709 | 0.4799 |
| 7 Nm | 1.0015 | 0.765 |

The device, system and method according to the present application quantify and evaluate the joint rotational laxity for clinical use, especially for orthopedics physicians to examine the knee rotational laxity of patients suffering from knee ligamentous injury, and to monitor their rehabilitation progress during follow-up consultations. Beside clinic, this device, system and method are also applicable for the use in field, physiotherapy treatment room and operation theater. The target user groups will be mainly the orthopedic surgeons, but also the general physicians, physiotherapists, sport biomechanics researchers as well as coaches and team physicians.

It is understood for those skilled in the art that embodiments described herein are illustrative, but not limited. Technical features disclosed in various embodiments can be combined in any appropriate ways. Various modifications and variations of the described embodiments can be made within the scope and spirit of the present application.

What is claimed is:

1. A device for measuring rotation of a first joint of an object, comprising:
an orthosis for immobilizing a second joint of the object, the second joint being connected to the first joint by a bone of the object;
a torque sensor fixed to the orthosis for monitoring a value of torque applied to the second joint, the applied torque being transferred to the first joint via the bone; and
a motion sensor for tracking a rotational displacement of the bone occurring under the applied torque.

2. The device of claim 1, wherein the motion sensor is arranged so that a longitudinal axis of the motion sensor is along the bone.

3. The device of claim 1, further comprising a handle fixed to the torque sensor for applying the torque.

4. The device of claim 1, wherein the torque is applied in a direction such that the first joint rotates inwardly or outwardly with the bone.

5. The device of claim 1, wherein the torque sensor is a load cell.

6. The device of claim 1, wherein the motion sensor is fixed to the torque sensor.

7. The device of claim 1, wherein the motion sensor is an electromagnetic tracking sensor.

8. The device of claim 1, wherein the first joint is a knee joint and the second joint is an ankle joint.

9. A method for measuring rotation of a first joint of an object, comprising:
immobilizing a second joint of the object, the second joint being connected to the first joint by a bone of the object;
monitoring a value of torque applied to the second joint, the applied torque being transferred to the first joint via the bone; and
tracking a rotational displacement of the bone occurring under the applied torque.

10. The method of claim 9, further comprising applying a torque to the second joint before monitoring the value of torque applied thereto.

11. The method of claim 10, wherein the torque is applied in a direction such that the first joint rotates inwardly or outwardly with the bone.

12. The method of claim 9, wherein the first joint is a knee joint and the second joint is an ankle joint.
